(19) United States
(12) Patent Application Publication
(10) Pub. No.: US 2009/0311211 A1
(43) Pub. Date: Dec. 17, 2009

(54) Aqueous Dispersions Of Polyorganosiloxanes Containing Urea Groups

(75) Inventors: Harald Chrobaczek, Augsburg (DE); Rule Niederstadt, Augsburg (DE); Theodor Angele, Wertingen (DE); Simpert Lüdemann, Bobingen (DE)

Correspondence Address: Huntsman Textile Effects Germany G;Legal Department, 10003 Woodloch Forest Drive, The Woodlands, TX, 77380, US

(73) Assignee: Huntsman Textile Efficts (Grmany) GmbH, Langweid (DE)

(21) Appl. No.: 11/721,570

(22) PCT Filed: Nov. 19, 2005
(86) PCT No.: PCT/EP2005/012414

§ 371 Date: Jun. 13, 2007

(30) Foreign Application Priority Data
Dec. 14, 2004 (EP) 04029522.2

(51) Int. Cl. A61K 8/898; A61Q 5/00; D06M 15/643

(52) U.S. Cl. 424/70.122; 252/8.61

(57) ABSTRACT

Polyorganosiloxanes containing urea groups can be obtained in the form of aqueous dispersions by firstly dissolving them in an organic solvent, then dispersing this solution with water with co-use of auxiliaries, subjecting the mixture to homogenization and then at least partially removing the organic solvent. The aqueous dispersions prepared in this way are suitable as additive for detergents, fabric softeners, haircare compositions, shampoos and for textile finishing.

The invention relates to aqueous dispersions which comprise polysiloxanes with urea groups and can be prepared by means of a specific process. It further relates to the use of such dispersions.

Aqueous dispersions of polyorganosiloxanes are used widely in the form of emulsions (“silicon emulsions”) of liquid or flowable polysiloxanes, thus, for example, in textile finishing, where textile sheet materials such as fabric are treated with these emulsions. Such polyorganosiloxanes are often used in the form of aqueous emulsions because this results in cost and environmental advantages compared with using them as the solution in organic solvents.

Aqueous dispersions of specific polyorganosiloxanes, their preparation and their use are described, inter alia, in WO 88/08436.

Also known from the prior art are polyorganosiloxanes containing urea groups, e.g. from EP-A 455 585.

DE-A 1 570 576 describes emulsions of polyorganosiloxanes which have salt-like groups.

EP-A-1 226 813 reveals the use of dispersions for haircare which comprise specific polyorganosiloxanes.

The preparation of silicone emulsions is also described in DE-A 197 22 403 and in German patent specification No. 900 018.

The prior art reveals a large number of methods as to how aqueous emulsions of such polysiloxanes which contain no urea groups can be prepared. Thus, for example, polyorganosiloxanes which are liquid at room temperature can be converted into stable aqueous dispersions by a direct method by mixing these siloxanes with dispersant and then dispersing the mixture in water or by firstly preparing a mixture of water and dispersant and dispersing the polysiloxane in this mixture. Such aqueous dispersions can be used for a very wide variety of purposes. However, it has been found that the majority of the methods known from the prior art for the direct dispersion of polyorganosiloxanes in water are unsuitable for converting polyorganosiloxanes containing urea groups into stable aqueous dispersions. This is because such polyorganosiloxanes are in many cases not liquid and not flowable at room temperature, but are in the form of thermoplastic polymers, the direct dispersion of which in water by conventional methods meets with considerable difficulties or is not possible at all. On the other hand, polyorganosiloxanes containing urea groups have advantageous properties, meaning that it is desirable to use them for some fields of use and as far as possible in the form of aqueous dispersions.

The object of the present invention was to provide aqueous dispersions of polyorganosiloxanes containing urea groups which are preparable by a process which allows stable aqueous dispersions also of polyorganosiloxanes containing urea groups which are non-liquid and non-flowable at room temperature to be obtained. In addition, the object was to provide such dispersions of the specified type which are exceptionally suitable for use in detergents, fabric softeners, hair-washing compositions, haircare compositions and for the finishing of textiles.

The object was achieved by an aqueous dispersion which can be prepared by the following, successive process steps:
a) preparation of a solution of a polyorganosiloxane containing urea groups or a mixture of such polyorganosiloxanes in an anhydrous organic solvent, of which, at room temperature, not more than 5 g are soluble in 100 g of water,
b) mixing of the solution obtained in step a) with water using an auxiliary which is chosen from one or more dispersants, polyvinyl alcohol (PVA) and guar meal (GuM) and mixtures of 2 or more of these products.
c) homogenization of the mixture obtained in step b), preferably by means of high-pressure homogenization,
d) removal of 90% to 100% of the organic solvent used in step a).

The dispersions according to the invention are excellently suitable as additive for detergents and for fabric softeners. For this, they can be added to conventional detergents or conventional fabric softeners which are used by the end consumer, e.g. in the home.

It has been found that through the abovementioned use according to the invention of the dispersions as fabric softener, washed articles can be obtained, e.g. textiles, which have particularly advantageous properties, such as good crease recovery properties in conjunction with a pleasantly soft handle, i.e. properties which come under the term “appearance after washing”. Among others, cellulose articles which consist completely or to a considerable degree of cellulose fibers can be treated in this way.

If dispersions according to the invention are added to customary detergents or fabric softeners, then this is preferably in an amount such that 0.1 to 70% by weight of polyorganosiloxanes containing urea groups are present in the end product, preferably 1 to 50% by weight.

A further preferred use of dispersions according to the invention consist in adding them to conventional haircare compositions or hair-washing compositions such as shampoos. Through this, good shape stability can be imparted to the hair or to the hairstyle.

In addition, dispersions according to the invention can also be used for the treatment of textile sheet materials, preferably for the treatment of fabrics, Such a treatment can be carried out in the course of the finishing of textiles, which is carried out, with respect to time, after customary pretreatment and dyeing processes, but before manufacture. This can impart good shape stability and crease properties to the textiles. Customary products, as are known for textile finishing, can be added to the dispersions according to the invention, such as, for example, cellulose crosslinkers, fire retardants, etc. Dispersions according to the invention are highly suitable for the treatment of fabrics which consist entirely or to a considerable degree of cellulose fibers.

The use of dispersions according to the invention for textile treatment can be carried out by customary methods known to the person skilled in the art, e.g. by means of padding.

If dispersions according to the invention are to be used for the specified finishing of textile sheet materials, it is often advantageous to add a polyorganosiloxane which contains one or more epoxy groups in the molecule to the dispersions before using them. Such polyorganosiloxanes containing epoxy groups are commercially available products. Examples thereof are the products Wacker IM 35, Wacker Siliconöl M643, Wacker SLM 441112/A, Wacker OG-144 dest. and Wacker Epoxy-Öl OG 268 from Wacker-Chemie GmbH, Germany.

The polyorganosiloxane containing epoxy groups is added here to the dispersion according to the invention preferably in amounts such that 6 to 12 g of polysiloxane which contains epoxy groups are used per 100 g of dispersion according to the invention. This range from 6 to 12 g is based on 100% strength, undiluted polyorganosiloxane containing epoxy groups. This polyorganosiloxane can be added as an undiluted product or in the form of an aqueous dispersion.

The advantageous effect of an addition of polyorganosiloxane containing epoxy groups consists in the finished textiles often being given further improved properties with regard to “appearance after washing”, e.g. improvement in the non-iron property or the crease behavior. Highly suitable polyorganosiloxanes containing epoxy groups are those with an epoxy number from 0.300 to 0.450 (mol of epoxidel100 g).

To prepare dispersions according to the invention, use is made of a polyorganosiloxane which contains urea groups. A mixture of such polyorganosiloxanes may also be used. The use of a mixture of such siloxanes is the norm because during the synthesis of these polymers mixtures of molecules with varying molecular weight are produced. The mixtures can be used directly to prepare dispersions according to the invention. A polyorganosiloxane containing urea groups thus comprises at least one unit of the formula

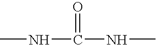

A plurality of such units may of course also be present.

These units can in each case be bonded to a silicon atom via an alkylene group, where this alkylene group may be interrupted by one or more secondary or tertiary amino groups.

Urea groups of the formula

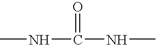

may be present on one or both chain ends of the polyorganosiloxane. In this case, the polyorganosiloxanes have, at one or both chain ends, preferably units of the formula (I)

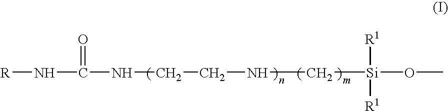

where m is a number from 1 to 4 and n has the value 0 or 1,
the radicals R¹, independently of one another, are in each case an alkyl group having 1 to 6 carbon atoms, preferably a methyl group, and where R may be an alkyl radical having 1 to 8 carbon atoms or a phenyl group, where this phenyl group may be substituted by one or more alkyl groups each having 1 to 4 carbon atoms.

However, the radical R in formula (I) is preferably a radical of the formula (II)

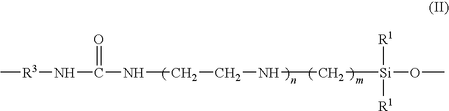

Here, the divalent radical R³ is bonded to the terminal nitrogen atom of formula (I) (to which in formula (I) the radical R is bonded).

In this embodiment, two linear polyorganosiloxane chains are joined at their ends via a bridge which contains two urea units

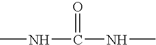

between which the divalent radical R³ is located.

In formula (II), m, n and R¹ have the meanings given above, R³ in formula (II) is a linear or branched alkylene radical having 2 to 10 carbon atoms or is a divalent aromatic radical, where this aromatic radical is preferably a radical which is derived from tolylene diisocyanate or diphenylmethane diisocyanate by removal of the two NCO groups.

Of very particularly good suitability for the preparation of dispersions according to the invention are polyorganosiloxanes containing urea groups in which at least one urea group of the formula

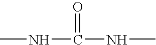

is located in a side chain of the polyorganosiloxane. Such preferably used polyorganosiloxanes thus contain one or more structural units of the formula (III)

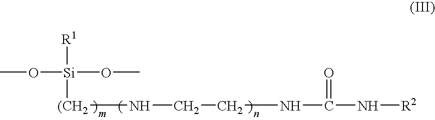

where R¹, m and n have the meanings given above and R² may be an alkyl radical having 1 to 8 carbon atoms or a phenyl group, where this phenyl group may be substituted by one or more alkyl groups each having 1 to 4 carbon atoms.

It is particularly preferred if the radical R² in formula (III) is a radical of the formula (IV)

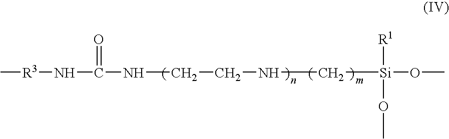

where the divalent radical R³ is bonded to the terminal nitrogen atom of formula (III) (to which in the formula (III) the radical R² is bonded).

In formula (IV), two polyorganosiloxane chains are thus joined together at their side chains via one or more bridges which contain urea groups. In formula (IV), m, n, R¹ and R³ have the meanings given above.

The described polyorganosiloxanes with urea groups can preferably be prepared by reacting polyorganosiloxanes which contain one or more radicals each having a terminal primary amino group with mono- or diisocyanates. In the process, the addition in each case of an —NH₂ group onto an —NCO group produces one or more urea units

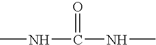

For this reaction, one or more types of polyorganosiloxanes and/or one or more types of mono- or diisocyanates can be used. It is also possible to use mixtures of mono- and diisocyanates.

The reaction of the primary amino groups of the polyorganosiloxane with isocyanate groups can take place under known conditions with which the chemist is familiar.

If, for the specified reaction, polyorganosiloxanes are used which contain radicals of the formula

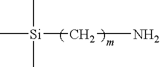

and no secondary or tertiary amino groups, then polyorganosiloxanes are produced which contain units of the formula (I) or of the formula (III) in which n=0. Products where n=1 can be prepared, on the other hand, by reacting polyorganosiloxanes with units of the formula (V)

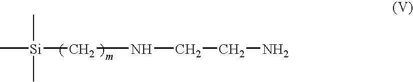

with isocyanates. Polyorganosiloxanes containing urea groups in which the units which contain

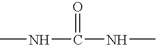

are present in side chains of the polymer can be prepared starting from aminofunctional polyorganosiloxanes which contain one or more primary amino groups in side chains.

If the aminofunctional polyorganosiloxanes are reacted with monoisocyanates RNCO or R²NCO, then polymers can be obtained in which structural units of the formula (I) or (III) are present in which R or R² is an alkyl radical having 1 to 8 carbon atoms or is an unsubstituted or substituted phenyl group. In this reaction use is thus made of monoisocyanates of the formula RNCO or R²NCO in which R or R² has the last-mentioned meaning.

However, to prepare dispersions according to the invention, particular preference is given to using polyorganosiloxanes which are formed by reacting aminofunctional polyorganosiloxanes with diisocyanates of the formula OCN—R³—NCO. In the polyorganosiloxanes containing urea groups formed in this process, two polyorganosiloxane chains are joined either at their ends or at their side chains by means of bridges which contain urea groups.

The diisocyanates used for this may be for example:

hexamethylene diisocyanate, isophorone diisocyanate, tolylene diisocyanate or diphenyl-methane 4,4′-diisocyanate.

The aminofunctional polyorganosiloxanes which are reacted with diisocyanates are preferably polydimethylsiloxanes in which one or more methyl groups are substituted by radicals of the formula (VI)

CH₂ₘNH—CH₂—CH₂ₙNH₂ (VI).

Such aminofunctional polysiloxanes are commercially available, e.g. from the companies Wacker or Dow Corning.

Suitable polyorganosiloxanes containing urea groups and their preparation are described in EP-A 455 585, U.S. Pat. No. 3,878,168 and WO 96/34030.

Of good suitability for the preparation of dispersions according to the invention are polyorganosiloxanes containing urea groups which are not liquid and not flowable at room temperature and which have a softening point or range above 50° C., or thermoplastic polysiloxanes containing urea groups. Especially in the case of such siloxanes, the preparation of stable aqueous dispersions by directly dispersing the polymers in water is difficult or cannot be carried out. On the other hand, however, these polyorganosiloxanes containing urea groups have the advantage that textiles finished with them have particularly good shape stabilities after washing processes.

Preferably, for the preparation of dispersions according to the invention, use is made of those polyorganosiloxanes in which no radicals are bonded to the silicon atoms other than urea groups or radicals containing urethane groups or alkyl groups having 1 to 6 carbon atoms or unsubstituted phenyl radicals. All of the end groups of the polyorganosiloxanes in which neither urea groups nor urethane groups are present are thus preferably groups of the formula

(R⁴)₃Si—O—

where all of the radicals R⁴, independently of one another, are an alkyl group having. 1 to 6 carbon atoms or the phenyl radical C₆H₅.

Polyorganosiloxanes containing urea groups which can be used to prepare dispersions according to the invention are commercially available.

Besides urea units, the polyorganosiloxanes containing urea groups can also additionally contain one or more urethane groups of the formula

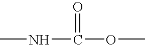

in which the terminal oxygen atom is bonded to a carbon atom. Such urethane groups can be formed by using polyorganosiloxanes which additionally contain hydroxy groups in the above-described reaction of aminofunctional polyorganosiloxanes with diisocyanates.

Aqueous dispersions according to the invention can be prepared by the process described below, it being necessary to observe the order a-b-c-d of the process steps.

### Step a):

In this process step, a solution of the polyorganosiloxane used or of the mixture of polyorgano-siloxanes in an organic solvent is prepared. It is also possible to use a mixture of two or more organic solvents. Such mixtures are likewise referred to below as “organic solvent”. If appropriate, the solution is prepared at elevated temperature. To prepare the solution in step a), either no siloxanes or polyorganosiloxanes other than polyorganosiloxanes containing urea groups of the type described above are used, or one or more further polyorganosiloxanes which contain no urea groups are used in the preparation of the solution according to step a). Of good suitability for this purpose are known polyorganosiloxanes which contain amino groups and are commercially available. The amount of such additionally used polyorganosiloxanes is preferably 1 to 50 g per 100 g of polyorganosiloxane containing urea groups.

It is advantageous if the solution prepared in step a) comprises 5 to 50% by weight of polyorganosiloxane, preferably 10 to 40% by weight. These amounts refer to the sum of all of the polyorganosiloxanes present.

After carrying out process steps a) to d) it is also possible to add other polysiloxanes to the dispersions according to the invention before the latter are further processed, e.g. standard commercial liquid aminofunctional polyorganosiloxanes or aqueous dispersions of such siloxanes.

The organic solvent used for step a) must be anhydrous. This does not mean that it must comprise no water at all, but the water content of this solvent should not exceed 2% by weight. The polyorganosiloxane used should also of course not comprise more than about 2% by weight of water.

Moreover, the solvent used should have only low solubility in water. At room temperature, this solubility should not exceed 5 g of solvent in 100 g of water.

Suitable solvents are higher alcohols, esters, ketones, e.g. dialkyl ketones, but also aromatic hydrocarbons, such as toluene or xylene provided they satisfy the conditions specified above and the polyorganosiloxane or polyorganosiloxane mixture used is soluble in the desired concentration in the solvent, where if necessary elevated temperature may be required for the dissolution.

With regard to process step d) (removal of at least 90% of the solvent), preference is given to using those solvents which can be readily removed from the dispersions prepared in step c). A simple method for this is distillation, optionally under reduced pressure. Solvents which can be removed in this way to at least 90% include solvents with a boiling point of less than 100° C. at room temperature, solvents which form an azeotrope with water or solvents which have a higher vapor pressure than water at reduced pressure. Particularly well-suited solvents are methyl isobutyl ketone and n-butyl acetate; both can be readily removed by distillation in step d) as azeotrope with water.

### Step b):

In process step b), the solution obtained after step a) is mixed with water. In this mixing operation, an auxiliary, as described below, is additionally used. The mixing can normally take place at room temperature. If appropriate, the solution of the polyorganosiloxane or the water or both have an elevated temperature. It is advisable to stir the mixture mechanically in order to aid the mixing operation.

The mixing is preferably carried out in quantitative ratios such that the finished aqueous dispersion obtained after carrying out step d) comprises 5 to 70% by weight, in particular 10 to 50% by weight, of polyorganosiloxanes containing urea groups. In addition, the finished dispersion according to the invention preferably comprises 0.5 to 10% by weight of an auxiliary of the type described below.

Before mixing the solution of the polyorganosiloxane with water, this auxiliary can be added to this solution. However, it is preferred to add the auxiliary to the water before it is mixed with the solution obtained in step a).

In step b), an auxiliary is used. This is chosen from one or more dispersants, polyvinyl alcohol (PVA) and guar meal (GuM). Also of suitability are mixtures which comprise one or more dispersants and additionally either PVA or GuM or which comprise, in addition to dispersant(s), both PVA and GuM. In addition, dispersants can be dispensed with entirely in a number of cases. In these cases, only PVA or only GuM or a PVA/GuM mixture is used.

In each of the specified cases, the total amount of auxiliary is preferably chosen such that the dispersion according to the invention obtained after step d) comprises 0.5 to 10% by weight of the auxiliary, irrespective of whether the auxiliary consists only of a single product from the group dispersant/PVA/GuM, or of a mixture which comprises 2 or more such products.

Suitable dispersants are known standard commercial surfaceactive products. Preference is given to using nonionic or cationic dispersants or a mixture of dispersants which are chosen from nonionic and cationic dispersants. The amount of dispersant or dispersant mixture can be in the range which is known to the person skilled in the art in the field of silicone emulsions. Preference is given to using amounts such that the finished dispersion according to the invention which is obtained after carrying out step d) comprises 0.5 to 10% by weight of dispersant or dispersant mixture. This latter statement applies if the auxiliary used consists exclusively of one or more dispersants.

Suitable nonionic dispersants are known standard commercial surface-active products, such as, for example, ethoxylated alcohols, ethoxylated fatty acids or ethoxylated fatty amines, each having linear or branched, saturated or unsaturated hydrocarbon radicals which contain 10-24 carbon atoms. Examples thereof are ethoxylated isotridecyl alcohols and ethoxylated stearic acid or oleic acid each having 6 to 12 polyoxyethylene units.

Suitable cation-active dispersants are, in particular, ammonium salts of the formula (VII).

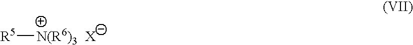

where R⁵ is a linear or branched, saturated or unsaturated hydrocarbon radical having 8 to 24 carbon atoms, all of the radicals R⁶ are, independently, hydrogen or an alkyl radical having 1 to 7 carbon atoms, which may be substituted by a hydroxy group, or is a radical R⁵. Preferably, two or three radicals R⁶ are a methyl group, an ethyl group or a 2-hydroxyethyl group. If appropriate, one or more of the radicals Re may also be a polyoxyethylene radical having 6 to 18 polyoxyethylene units.

The auxiliary can also comprise PVA besides dispersant(s) and/or GuM or consist only of PVA. In this case, preference is given to using PVA with an average molecular weight in the range from 25 000 to 100 000.

Besides dispersant(s) and/or PVA, the auxiliary can likewise also comprise GuM or consist only of GuM. For this, preference is given to using GuM which is characterized by the following data: viscosity of a 1% solution at 25° C.: 3.5-3.7 Pa.s (3500-3700 cP) average particle size: about 75·10⁻⁶ m. GuM is commercially available, e.g. from Worlée-Chemie GmbH, Germany. Guar meal (GuM) is a product known from the literature, see e.g. “Römpp Chemie Lexikon”, Georg Thieme Verlag Stuttgart, New York, 9th edition, 1990, page 1666.

In the case of dispersions according to the invention, PVA and GUM can bring about increased stability of the aqueous dispersion by acting as protective colloid.

A particularly preferred embodiment of dispersions according to the invention consists in, during their preparation in step b), using as auxiliary a mixture which comprises one or more cation-active dispersants and additionally PVA and/or GUM. Highly suitable cation-active dispersants here are the products already specified above.

### Step c):

In order to obtain stable aqueous dispersions of the polyorganosiloxanes containing urea groups, it is necessary to subject the mixture of polysiloxane, auxiliary and water obtained after step c) to a homogenization. Depending on the type and amount of the products used, this homogenization can, if appropriate, be carried out at room temperature.

However, it is preferably carried out at elevated temperature, e.g. in the range from 40-80° C. It is also advantageous to carry out the homogenization in the form of a high-pressure homogenization.

The high-pressure homogenization is preferably carried out at a pressure in the range from 50 to 1000 bar, a particularly favorable range is 50 to 350 bar. The pressure can be established and controlled in a targeted manner in a series of known apparatuses of the type described below.

Information on equipment and processes for high-pressure homogenization can be found in WO 99/15263.

Of suitability for carrying out the high-pressure homogenization according to process step c) are Gaulin instruments from Manton-Gaulin Corp. USA or APV-Deutschland GmbH, Lubeck, Germany, e.g. the models “Gaulin Homogenizer LAB 60-7 TBS” or the MC models from this company. Also suitable are instruments of the “Microfluidizer” series from Microfluidics, USA.

### Step d):

The last step of the process by which dispersions according to the invention can be prepared consists in removing 90% to 100% of the quantity of the solvent used for step a). For a number of application purposes, e.g. for use in detergents or fabric softeners or the use for hair-washing/haircare compositions, it is expedient to remove the solvent completely or virtually completely. in some cases, however, it may suffice or even be desired to remove only some of the solvent, e.g. in the case of use for coating purposes, such as coating of fabrics. Products which are prepared by the process described in the present patent application are thus aqueous dispersions for the purposes of the present invention even if they still comprise up to 10% of the amount of solvent which was used for step a). The partial or complete removal of the solvent can be carried out by known methods. Normally, a distillation is advantageous. By choosing suitable solvent properties it is possible, as described above, to facilitate distillative separation.

The invention is illustrated below by working examples.

### EXAMPLE 1

### According to the Invention

### Preparation of a Dispersion According to the Invention:

100 g of a polydimethylsiloxane which contains urea groups (TPS or TPSE 140 from Wacker GmbH, Germany) are dissolved in 300 g of methyl isobutyl ketone (MIBK) at about 80° C. 600 g of an aqueous dispersant solution are added to this solution. The pH of the mixture obtained is adjusted to a value of about 3.3 at 60° C. by adding 60% strength acetic acid.

The aqueous dispersant solution comprises about 550 g of water, 20 g of a mixture of a cation-active dispersant (quaternary ammonium salt), and a nonionogenic dispersant, and 20 g of 1,2-propylene glycol.

The mixture is then stirred, during which a preemulsion is formed; this is subjected to a high-pressure emulsification at about 50° C., during which a homogeneous dispersion is formed. MIBK is removed from this virtually quantitatively (as an azeotrope with water) by distillation under reduced pressure. This gives a slightly opalescent dispersion with a content of polysiloxane+dispersant of about 29% by weight.

### EXAMPLE 2

### According to the Invention

A solution of a polydimethylsiloxane containing urea units in MIBK is prepared and further processed as in example 1 except that instead of 20 g of a dispersant mixture, 22 g of a mixture of cation-active dispersant and polyvinyl alcohol (PVA) (Polyviol 25/140 from Wacker GmbH, Germany) is added. This gives an opalescent dispersion with about 24% by weight fractions excluding water.

### EXAMPLE 3

### According to the Invention

The procedure is as in example 2 but using 5 g of a mixture of cation-active dispersant and guar meal (GuM) (NOGU 4401 from Worlée, Germany) instead of 22 g of a mixture of cation-active dispersant and PVA. This gives a slightly opalescent dispersion.

### EXAMPLE 4

### Comparative Example not According to the Invention

Attempts are made to convert the polydimethylsiloxane containing urea groups which is used in the preceding examples into an aqueous dispersion by a customary method. Since this siloxane is of solid consistency, it is attempted to melt it as solid and then to disperse it or to add it as a solid to an aqueous dispersant solution and then to convert this mixture into an aqueous siloxane dispersion by heating.

Both attempts come to nothing: it is not possible to obtain an aqueous dispersion of the polysiloxane in this way.

### EXAMPLE 5

Fabric consisting of 100% cotton is treated by means of a padding process with the dispersions obtained as in examples 1 to 3 to which 4% by weight of a standard commercial cellulose crosslinker (KNITTEX® FEL from Ciba Spezialitättenchemie Pfersee GmbH) has been added. Drying of the fabric is then carried out at 110° C./10 min and condensation at 150° C./5 minutes. The fabric has good crease behavior, which is superior to that of fabrics which are treated with a conventional silicone emulsion which comprises cellulose crosslinker under identical conditions.

### EXAMPLE 6

Example 5 is repeated, adding also about 30 parts by weight of polysiloxane dispersion containing epoxy groups (OG 144 from Wacker GmbH, Germany) to the dispersions according to the invention as in example 1 to 3 per 100 parts by weight of dispersion according to the invention. With the 3 dispersions prepared in this way as well, similarly good fabric properties are achieved as in example 5.

1. An aqueous dispersion preparable by the following, successive process steps:
a) preparation of a solution of a polyorganosiloxane containing urea groups or a mixture of said polyorganosiloxanes in an anhydrous organic solvent, of which, at room temperature, not more than 5 g are soluble in 100 g of water,
b) mixing of the solution obtained in step a) with water using an auxiliary which is selected from the group consisting of one or more dispersants, polyvinyl alcohol (PVA), guar meal (GuM) and mixtures thereof.
c) homogenization of the mixture of polyorganosiloxane, auxiliary and water obtained in step b), and
d) removal of 90% to 100% of the quantity of organic solvent used in step a).

2. The dispersion as claimed in claim 1, characterized in that, in step a), a polyorganosiloxane containing urea groups is used which has been prepared by reacting a polyorganosiloxane containing one or more primary amino groups with mono- or diisocyanate or with a mixture of mono- and diisocyanate.

3. The dispersion as claimed in claim 1, characterized in that the solution prepared in step a) comprises 5 to 50% by weight of polyorganosiloxane.

4. The dispersion as claimed in claim 1, characterized in that, in step b), the auxiliary used is a nonionic or a cationic dispersant or a mixture of said dispersants.

5. The dispersion as claimed in claim 1, characterized in that, in step b), the auxiliary used is a mixture which comprises one or more cation-active dispersants and additionally PVA and/or GuM.

6. The dispersion as claimed in claim 1, characterized in that step b) is carried out such that the auxiliary has been added to the water before mixing the solution obtained in step a) with water.

7. The dispersion as claimed in claim 1, characterized in that step c) is carried out at a pressure in the range from 50 to 1000 bar.

8. The dispersion as claimed in claim 1, characterized in that it comprises 0.5 to 10% by weight of an auxiliary.

9. The dispersion as claimed in claim 1, characterized in that it comprises 5 to 70% by weight of polyorganosiloxane containing urea groups.

10. An additive for detergents or fabric softeners comprising the aqueous dispersion of claim 1.

11. A haircare composition comprising the aqueous dispersion of claim 1.

12-14. (canceled)

15. A method for the treatment of textile sheet materials in the course of textile finishing comprising applying the aqueous dispersion of claim 1 to the textile sheet materials.

16. The method of claim 15, characterized in that the aqueous dispersion further comprises a polyorganosiloxane which contains one or more epoxy groups.

17. The method of claim 16, characterized in that the polyorganosiloxane which contains one or more epoxy groups has an epoxy number of from 0.300 to 0.450 mol of epoxide/100 g.
